(19) United States
(12) Patent Application Publication
(10) Pub. No.: US 2006/0183247 A1
(43) Pub. Date: Aug. 17, 2006

(54) Detection method for specific biomolecular interactions using fret between metal nanoparticle and quantum dot

(75) Inventors: Hak-Sung Kim, Daejeon (KR); Eunkeu Oh, Daejeon (KR); Mi-Young Hong, Daejeon (KR); Dohoon Lee, Seoul (KR); Sunghun Nam, Anyang (KR)

Correspondence Address: STAAS & HALSEY LLP, SUITE 700, 1201 NEW YORK AVENUE, N.W., WASHINGTON, DC, 20005, US

(73) Assignee: KOREA ADVANCED INSTITUTE OF SCIENCE AND TECHNOLOGY, Daejeon (KR)

(21) Appl. No.: 11/355,069

(22) Filed: Feb. 16, 2006

(30) Foreign Application Priority Data
Feb. 16, 2005 (KR) 2005-0012865
Feb. 8, 2006 (KR) 2006-0012213

(51) Int. Cl. G01N 33/551

(52) U.S. Cl. 436/524; 977/900

(57) ABSTRACT

The present invention relates to (1) a method for detecting a specific binding for bio-molecules; (2) a method for detecting a presence of target molecules within a specimen; and (3) a method for measuring a quantity of target molecules existing in a specimen, in which a pair of energy donor and energy acceptor displaying a FRET phenomenon is used to guide a specific binding between a pair of bio-molecules. According to the present invention, the process is conducted rapidly and easily without a label so as to screen a biochemical substance inhibiting a specific binding between a pair of bio-molecules in an ultra-high speed and measure a quantity of the substance and thereby, it can be applied to develop a novel drug. Further, this method can be used to analyze a characteristic such as change of the amount of carbohydrates in a glycoprotein derived from various cells as a drug candidate and thereby, is applicable for a quality control of proteins etc.

### BACKGROUND OF THE INVENTION

### Field of the Invention

The present invention relates to a novel method for detecting a target molecule, more particularly to a method for screening target molecules and measuring a quantity of target molecules in an ultra-high speed by using resonance energy transfer between metal nano-particle and quantum dot.

### BACKGROUND OF THE RELATED ART

Nano-particle, a main material of nano-technology in 1˜100 nm of size scale has a wide applicability in various research fields including physics, chemistry, biology, and medical science etc. Precisely, it is useful for a reaction catalyst, a building block for nano-patterning use, a molecular tag for diagnosis, and a nano-sized factor vehicle etc. For this purpose, several kinds of nano-particles are being manufactured by using metals, nonmetals, and semi-conductors etc. (Murphy C., J. Anal. Chem., A-Pages 74: 520A, 2002).

The nano-particles are spotlighted in several applications including bio-engineering areas, because they have physico-chemical or optical characteristics peculiar according to the kinds or sizes of core metals. Precisely, gold nano-particles in several ˜ several tens nano-meters represent a surface plasmon resonance band at near 520 nm and change their wavelength according to their adjacent environment or other material attached onto the nano-particle. Semi-conductor nano-particles are excited by UV irradiation and display a characteristic of quantum dot so as to emit visible light in the wide range of wavelength when reaching less than 10 nm of particle size.

Fluorescence resonance energy transfer (hereinafter, referred to as “FRET”) is reported to occur between quantum dot and gold nano-particle or dyes different in the wavelength. Therefore, a lot of researches have been accomplished actively in bio-engineering fields in order to analyze a cell image and a protein interaction etc. by using this FRET. (Alivisatos A. P., Nat. Biotechnol. 22:47, 2004). However, it is unclear that the FRET occurrence between gold nano-particle and quantum dot might be applied to measure a specific binding of bio-molecules or detect/screen its inhibitor.

In conventional methods, ELISA, microarray, electrochemical signal sensing, calorimetric sensing by aggregation between gold nano-particles and the like have been attempted to detect inhibitors for bio-sensing that interferes a specific binding between bio-molecules (hereinafter, referred to as “target molecule”). However, there are several disadvantages. These techniques require a lot of time and efforts, because they should label a specimen, amplify a signal by an additional reaction, or need a specimen in a large scale. Therefore, it is enforced to develop a novel method for detecting a target molecule, in which the procedure is conducted rapidly and easily even with a small amount so as to screen several substances simultaneously without a label.

For example, the detection of target molecules also includes a detection of glycoproteins. Glycosylation is one of post-translational modifications (PTMs) in proteins produced by in vivo gene expression like phosphorylation. The glycosylation is reported to play an important role to regulate a protein activity, solubility, resistance to degradation, immune reaction and signal transmission etc. (Varki A., Glycobiology, 3: 97-130, 1993). The protein is classified to have a different property by the kind and amount of carbohydrates combined. This processing depends upon cellular environment and status of cell growth or the kind and mutation of cells. Especially, the glycoprotein is reported to influence a pharmaceutical efficacy according to the degree or kind of carbohydrates attached when developing a novel drug. Therefore, it is urgently required to develop an efficient method for analyzing a glycoprotein.

Unfortunately in conventional techniques, the glycoprotein is characterized by the process as follows: (1) separating carbohydrates from a glycoprotein through a chemical reaction or enzymatic reaction; and (2) measuring a molecular weight of resulting protein to estimate its amount of carbohydrates. In this procedure, several methodologies including SDS-PAGE, Bio-LC and MALDI-TOF/MS etc. are applied to discriminate the molecular weight. Disadvantageously, they need a lot of time and efforts. Further, this technique that analyzes the amount of carbohydrates by measuring a molecular weight is too problematic to apply for a quality control or drug screening in a large scale.

### SUMMARY OF THE INVENTION

The object of the present invention is to provide to a method for screening target molecules and measuring a quantity of target molecules rapidly and easily by using resonance energy transfer between metal nano-particle and quantum dot.

The other object of the present invention is to provide to a method for detecting a glycoprotein and measuring a quantity of glycoproteins rapidly and conveniently by using fluorescence resonance energy transfer between metal nano-particle and quantum dot.

### BRIEF DESCRIPTION OF THE DRAWINGS

The above and other objects, features and other advantages of the present invention will be more clearly understood from the following detailed description taken in conjunction with the accompanying drawings, in which;

FIG. 1 depicts the emission spectrum of quantum dot showing a specific binding between gold nano-particle conjugated with biotin as a bio-molecule 1 and quantum dot conjugated with streptavidin as a bio-molecule 2;

FIG. 2 depicts the change of emissions according to the concentration of avidin in the solution of BG nano-particle and SQ dot;

FIG. 3 depicts the conceptual diagram of mechanism that the emission of quantum dot increases as the specific binding between BG nano-particle and SQ dot is inhibited by avidins (SA: streptavidin, QD: quantum dot, AV: avidin);

FIG. 4 depicts the electron microscopy showing a specific binding between BG nano-particle and SQ dot and its inhibition by avidins;

FIG. 5 depicts the change of emissions according to the concentration of glycoprotein in the solution of gold nano-particle conjugated with lectin as a bio-molecule 1 and quantum dot conjugated with carbohydrate as a bio-molecule 2;

FIG. 6a depicts the analysis of emission change according to the amount of carbohydrates by using a neoglycoprotein different in its amount of carbohydrates;

FIG. 6b depicts the analysis of emission change in a mini-well according to the amount of carbohydrates by using a neoglycoprotein different in its amount of carbohydrates.

### DETAILED DESCRIPTION OF THE PREFERRED EMBODIMENT

In order to achieve the objects, the present invention provides (1) a method for detecting a specific binding for bio-molecules; (2) a method for detecting a presence of target molecules within a specimen; and (3) a method for measuring a quantity of target molecules existing in a specimen, in which a pair of energy donor and energy acceptor displaying a FRET phenomenon is used to guide a specific binding between a pair of bio-molecules.

Fluorescence resonance energy transfer (FRET) is a phenomenon that if absorbing energy from outside, an energy donor, a shorter wavelength dye transfer excitation energy radiationlessly to an energy acceptor, a longer wavelength excitation dye, resulting in emitting a longer wavelength light from acceptor instead of a shorter wavelength light from donor. In order to generate fluorescence in the acceptor or extinct light in the donor by the FRET occurrence, both energy donor and acceptor should be placed within a very short distance. If the donor approaches the acceptor in a predetermined distance, the donor, a fluorescent substance emitting light having a particular wavelength transits the light energy irradiated from outside toward the acceptor without radiation. As a result, the donor decreases emission in its intrinsic wavelength and the acceptor increases emission in its intrinsic range of wavelength after absorbing energy from the donor.

The present invention is based upon a technique for immediately judging optically whether the FRET occurs or not, in which a pair of energy donor and energy acceptor possible to induce a FRET phenomenon are blended and reacted with bio-molecules respectively in order to detect a specific binding of bio-molecules.

Precisely, a donor and an acceptor conjugated respectively with a pair of specific-binding bio-molecules are reacted in a solution and thus, placed near each other so as to transfer luminescence from the donor to the acceptor. In this process, the FRET phenomenon occurs so that fluorescence (or luminescence) disappears from the donor and appears again from the acceptor. On the other hand, the acceptor may not radiate even after absorbing energy from the donor, if it does not have the property of fluorescence or luminescence. Only the donor loses emission in its own range of wavelength by the FRET, even though the acceptor does not radiate when the donor and the acceptor approach within a predetermined distance. (This invention belongs to this case)

Hereinafter, the present invention will be described more clearly as follows.

In the first embodiment, the present invention provides a method for identifying whether a pair of bi-molecules unclear in their correlation binds specifically or not.

Precisely, the method for detecting a specific binding for a pair of bio-molecules, which comprises steps as follows: (1) selecting a pair of energy donor and energy acceptor that displays a FRET phenomenon when approaching in a predetermined distance, as a metal nano-particle and a quantum dot respectively; and then, conjugating a pair of bio-molecules independently to the metal nano-particle and the quantum dot; and (2) blending a bio-molecule conjugated metal nano-particle (hereinafter, referred to as “BM particle”) and a bio-molecule conjugated quantum dot (hereinafter, referred to as “BQ dot”) resulted above in a liquid state; and then, identifying an induction of the FRET phenomenon by using a fluorescence assay, is provided.

Preferably, the metal nano-particle is selected from a group comprising gold nano-particle, silver nano-particle and platinum nano-particle. In the Examples of the present invention, the gold nano-particle is utilized, but it is natural to adopt silver nano-particle and platinum nano-particle, because silver and platinum also have surface plasmon resonance band (SPB) like gold.

In the description of the present invention, “bio-molecule 1” and “bio-molecule 2” are artificial terms to simply divide a pair of biological molecules (namely, 2 molecules). Accordingly, a bio-molecule 1 conjugated metal nano-particle (hereinafter, referred to as “B1M particle”) and a bio-molecule 2 conjugated quantum dot (hereinafter, referred to as “B2Q dot”) is substantially the same concept with a bio-molecule 2 conjugated metal nano-particle (B2M particle) and a bio-molecule 1 conjugated quantum dot (B1Q dot). Further, “interaction” between bio-molecules means an interaction by a specific binding between bio-molecules. Accordingly, it is substantially the same concept with “specific binding”. The bio-molecules can interact in various modes such as DNA-DNA, DNA-protein, protein-ligand, protein-protein, and antibody-antigen.

Preferably, the pair of bio-molecules can be selected from a group comprising genetic material or pseudo-genetic material including DNA, RNA and PNA etc., proteins including antigen and antibody etc., glycoproteins and carbohydrates. In addition, the bio-molecules can be enzyme and its substrate, enzymatic inhibitor or cofactor and the like. However, it is natural that the bio-molecules are not limited within the above-mentioned items and includes all the molecules, if possible to make a coupling.

According to the first embodiment of the present invention, it is convenient to identify whether a pair of (two) bi-molecules unclear in their correlation binds specifically or not.

In the second embodiment, the present invention provides a method for detecting a target molecule interfering a specific binding between a pair of bi-molecules further to screen an inhibitor.

Precisely, the method for detecting a target molecule in a specimen, which comprises steps as follows: (1) selecting a pair of energy donor and energy acceptor that displays a FRET phenomenon when approaching in a predetermined distance, as a metal nano-particle and a quantum dot respectively; then, conjugating a pair of bio-molecules (a bio-molecule 1 and a bio-molecule 2) independently at the metal nano-particle and the quantum dot; and preparing a bio-molecule 1 conjugated metal nano-particle (B1M particle) and a bio-molecule 2 conjugated quantum dot (B2Q dot); (2) blending the resulting B1M particle and the resulting B2Q dot in a liquid state; and then, identifying a specific binding for the B1M particle and the B2Q dot, that is to say whether sustaining the specific binding or not; and (3) blending the B1M particle and the B2Q dot with a specimen; and then, analyzing a change of the FRET occurrence by using a fluorescence assay.

The bio-molecule 1 and the bio-molecule 2 and the metal nano-particle are defined as described in the first embodiment of the present invention.

The bio-molecules can interact to each other in various modes such as DNA-DNA, DNA-protein, protein-ligand, protein-protein, and antibody-antigen. In contrast, other bio-molecules including ligands or proteins, genes, drugs, metal ions, cofactors like vitamins may interfere a specific binding between a pair of bio-molecules and acts as an inhibitor. These inhibitory bio-molecules are defined as the target molecules in the present invention.

The target molecule that may interact with a pair of specific-binding bio-molecules or interfere their specific binding is added to a mixture of the B1M nano-particle and the B2Q dot and thereby, obstructs the specific reaction of the bio-molecules so that the metal nano-particle and the quantum dot cannot approach within a distance possible to display a FRET occurrence. As a result, the quantum dot does not extinct its intrinsic emission because the FRET does not occur.

According to the second embodiment of the present invention, it is possible to rapidly identify whether a target molecule interfering a specific binding between a pair of bio-molecules binding specifically (further, interacting) in a specimen containing various bio-molecules. The specific binding of the bio-molecules is correlated with a signal transmission and therefore, can be used to treat various diseases by a proper regulation. That is to say, this method can be applied to screen novel drugs that treat or prevent diseases caused by the specific binding between particular bio-molecules. For example, several inhibitors blocking the reactivity of phosphate di-esterase-5 (PED-5) on its substrate is being screened to develop an impotence drug. In addition, other substance interfering a binding of histamine and its donor is being researched to commercialize a therapeutic drug of allergy.

In the third embodiment, the present invention provides a method for measuring a quantity (or concentration) of already-known target molecules in a specimen.

Precisely, the method for measuring a quantity of target molecules in a specimen, which comprises steps as follows: (1) selecting a pair of energy donor and energy acceptor that displays a FRET phenomenon when approaching in a predetermined distance, as a metal nano-particle and a quantum dot respectively; then, conjugating a pair of bio-molecules (a bio-molecule 1 and a bio-molecule 2) independently at the metal nano-particle and the quantum dot; and preparing a bio-molecule 1 conjugated metal nano-particle (B1M particle) and a bio-molecule 2 conjugated quantum dot (B2Q dot); (2) blending the B1M particle and the B2Q dot in a liquid state; and then, identifying a specific binding for the B1M particle and the B2Q dot, whether sustaining the specific binding or not; and (3) blending the B1M particle and the B2Q dot with a specimen containing a target molecule inhibiting a specific binding between the bio-molecule 1 and the bio-molecule 2; and then, analyzing a degree of the change in the FRET phenomenon by using a fluorescence assay, is provided.

The inhibition of the FRET occurrence between the B1M nano-particle and the B2Q dot is measured according to the concentration of target molecules in order to make a standard curve. Then, the FRET inhibition is estimated in a specimen so as to calculate the concentration of target molecules.

The bio-molecule 1 and the bio-molecule 2 and the metal nano-particle are defined as described in the first embodiment of the present invention.

In the first embodiment to the third embodiment of the present invention, the metal nano-particle and the quantum dot can be modified by several processings and then, conjugated with bio-molecules. The modification of the metal nano-particle and the quantum dot and the conjugation of bio-molecules may be accomplished by conventional processes. (Chem. review, 2004, pp 293-346 Marie-Christine Daniel & Didier Austruc)

The B1M nano-particle and the B2Q dot can be purchased among commercially-available products. Otherwise, they can be manufactured before use, depending upon requirements.

In the processings, the metal nano-particle or the quantum dot is modified on the surface by using a hydrophobic or hydrophilic functional group such as hydroxyl group (—OH), carboxylic group (—COOH), amine group (—NH₂), and thiol group (—SH). Then, the above-mentioned functional group and the functional group of target molecules are reacted with a physical bond by electrostatic force, hydrophobic interaction or van der Waals force; or a chemical bond such as covalent bond or metallic bond.

When attaching a bio-molecule onto a metal nano-particle, it is liable to decrease an electrostatic repulsion between metal nano-particles, reduce solubility and stability of nano-particles and increase a non-specific binding when detecting a target molecule due to ineffective modification. In order to settle such a problem, the metal nano-particle should be modified stably on the surface during or after preparing the nano-particle. In addition, several procedures are recommended in order to guarantee the stability while binding a bio-molecule. (Chem. review, 2004, pp 293-346 Marie-Christine Daniel & Didier Austruc)

Precisely in the Examples of the present invention, the metal nano-particle is stabilized by a secondary modification to increase its water solubility with dendrimers. In addition, the metal nano-particle is secured in its stability and solubility by preventing an aggregation between metal nano-particles. The metal nano-particle is reacted with an excessive amount of bio-molecules so as to retain the bio-molecules on the surface sufficiently and further, treated to reduce the non-specific binding caused by the ineffective modification maximally.

As described in the Examples, when being secondarily modified on the surface with avidins, the quantum dot is first conjugated with biotins and then, conjugated with avidins by using a specific binding of avidin and biotin so as to reduce a non-specific binding. In addition, when being modified with an amine group or a carboxylic group, the quantum dot can be conjugated with bio-molecules by using a variety of linkers and functional groups exposed on the surface through a chemical bond such as covalent bond, hydrogen bond or metallic bond and a physical bond by electrostatic force, hydrophobic interaction or van der Waals force.

The present inventors have examined and confirmed whether this process might detect a target molecule (for example, an inhibitor interfering a specific binding) and measure its quantity by using avidin and glycoprotein effectively as follows (See Examples).

Above all, in order to examine the detection of target molecules, the avidin-biotin binding is chosen because it is well-known to have the highest reactivity among interactions of protein-ligand and then, avidins are detected. For this purpose, a gold nano-particle conjugated with biotin as a bio-molecule 1 (hereinafter, referred to as “BG nano-particle”) and a quantum dot conjugated with streptavidin having specificity with biotin as a bio-molecule 2 hereinafter, referred to as “SQ dot”) are used.

In order to prevent a non-specific binding between gold nano-particle and quantum dot, the gold nano-particle is modified with dendrimers and then conjugated with biotins. The commercial quantum dot that is composed of CdSe at the core region and ZnS around the shell and conjugated with streptavidins is purchased from Quantum Dot Corporation.

The resulting fluorescence decreases at the SQ dot when the BG nano-particle exists. Therefore, it is observed that the biotin of gold nano-particle and the streptavidin of quantum dot are bound specifically. In addition, when adding avidins as a target molecule, the emission of quantum dot increases proportionally according to the concentration. Therefore, it is concluded that the avidin can be detected by measuring the ratio of emissions at the quantum dot and further, quantitated.

In order to measure a quantity of target molecules according to the present invention, a gold nano-particle conjugated with a lectin (hereinafter, referred to as “LG nano-particle”) as a bio-molecule 1 and a quantum dot conjugated with a dextran as a bio-molecule 2 (hereinafter, referred to as “DQ dot”) are chosen. The dextran is a carbohydrate composed of polymerized glucose binding specifically with lectin. For the target molecule, glycoprotein having carbohydrates binding with lectins is utilized to perform an assay.

### EXAMPLES

Practical and presently preferred embodiments of the present invention are illustrated as shown in the following Examples.

However, it will be appreciated that those skilled in the art, on consideration of this disclosure, may make modifications and improvements within the spirit and scope of the present invention.

The excitation wavelength for exciting a quantum dot is available in overall range of UV wavelength and is a well-known advantage of semi-conductor quantum dot. In following Examples, the quantum dot is excited at 440 nm of wavelength and its fluorescence spectrum is measured at 605 nm.

### Example 1

### Analysis of Avidin by FRET between BG Nano-Particle and SQ Dot

(1) Preparation of BG Nano-Particle

In order to prepare the BG nano-particle as a target molecule, 0.1 g of HAuCl₄.3H₂O was dissolved in 10 ml of DDW (Double distilled water) to prepare a gold chloride solution. Then, 6 mg of tetraoctylammonium bromide was dissolved in 3 ml of toluene. The resulting solution was added to the gold chloride solution and stirred vigorously at room temperature for 30 minutes. While a phase transition occurred, the gold chloride contained in a soluble layer rose up to an organic layer, changing its yellow color to a red and the organic layer was isolated. Then, 5 mg of 11-mercaptoundecanoic acid and 5 mg of 1-octanethiol were dissolved in 10 ml of toluene, added to the organic layer, and stirred strongly at room temperature for 5 minutes. After that, 2 mg of NaBH₄ was dissolved in 10 ml of DDW and dropped slowly. The mixture was stirred at room temperature for 2 hours. Then, a toluene layer was separated and centrifuged. The resulting precipitate was dissolved in 10 ml of ethanol again to prepare deep-brown gold nano-particles. When performing an UV/Vis spectroscopy, the gold nano-particles showed a slightly higher absorbance than adjacent bands at approximately 500˜520 nm of wavelength by surface plasmon resonance. Before forming nano-particles, a typical peak by surface plasmon resonance might not be observed at 500˜540 nm through an UV/Vis spectrum. As a result, it is confirmed that the gold nano-particle is prepared successfully.

After that, 1 ml of the gold nano-particle prepared by the above-mentioned procedure was mixed with 1 ml of 20% methanol solution of polyamidoamine (PAMAN), stirred and reacted at room temperature for a day. After reacting, the solution was centrifuged to discard non-reactive dendrimers. The resulting precipitate was dissolved in 1 ml of DDW. Then, 10 mg of N-hydroxysuccinimide-biotin (NHS-biotin) was added, stirred for an hour and centrifuged to produce BG nano-particles (Biotin-AuNPs).

(2) Detection of Specific Binding between BG Nano-Particle and SQ Dot

In order to examine a specific binding between SQ dot and BG nano-particle prepared in the Example 1(1), the SQ dot (SA-QDs) specifically binding with biotins was purchased from Quantum Dot Corporation (commercial name: Qdot605 streptavidin conjugates). The SQ dot is composed of CdSe at the core region and ZnS around the shell. It has a rod shape in 10˜15 nm of length and 5 nm of width and includes 15˜25 of streptavidins per quantum dot at the utmost shell. FRET fluorescence was observed with a fluorometer.

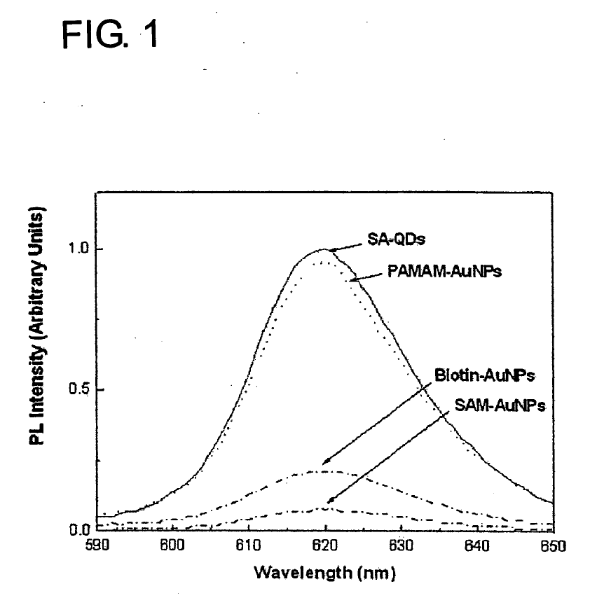

In order to detect a specific binding, 1 μM of BG nano-particle and 1 nM of the SA-QDs were blended and stirred for an hour to prepare an experimental group (Biotin-AuNPs). For a control group, gold nano-particles modified only with dendrimers on the surface (PAMAN-AuNPs) was blended with SQ dot and prepared with the same concentration. The emissions (fluorescences) of quantum dots were measured in two specimens of the experimental solution and the control solution. The result is illustrated in FIG. 1.

In FIG. 1, the SA-QDs indicates one emission spectrum corresponding to the SQ dot only. The control group adding the PAMAM-AuNPs remains a high emission of quantum dot because the energy transition of fluorescence did not occur. As predicted above, the gold nano-particle without conjugated biotin did not bind with the SA-QDs. As described in FIG. 1, it is concluded that the fluorescence of quantum dot is decreased by a specific binding between biotin and streptavidin in the experimental group (Biotin-AuNPs) because the biotin-AuNPs are bound with the SA-QDs so as to display FRET. As a consequence, this emission of quantum dot decreased more remarkably than that of the SA-QDs specimen.

(3) Analysis of Avidin According to Concentration by Using FRET Between BG Nano-Particle and SQ Dot

Avidin interfering a specific binding of streptavidin and biotin was adopted as an inhibitor and its inhibitory activity according to concentrations was measured quantitatively.

In detail, 100 nM solution of the BG nano-particle prepared in the Example 1 (biotin-AuNPs) was blended with the avidin. The mixture was adjusted to have 0 nM, 3 nM, 6 nM, 15 nM, 30 nM, 60 nM, 150 nM, 300 nM, 600 nM, 1.5 μM, and 2.5 μM of avidin concentration respectively and reacted at room temperature for an hour. Each reactant was mixed with the SA-QDs, while finally adjusting to 1 nM and reacted again at room temperature for an hour. At this moment, DDW was utilized. In order to prevent a non-specific binding and maintain a stability of quantum dot, bovine serum albumin (BSA) was added to reach 100 nM respectively.

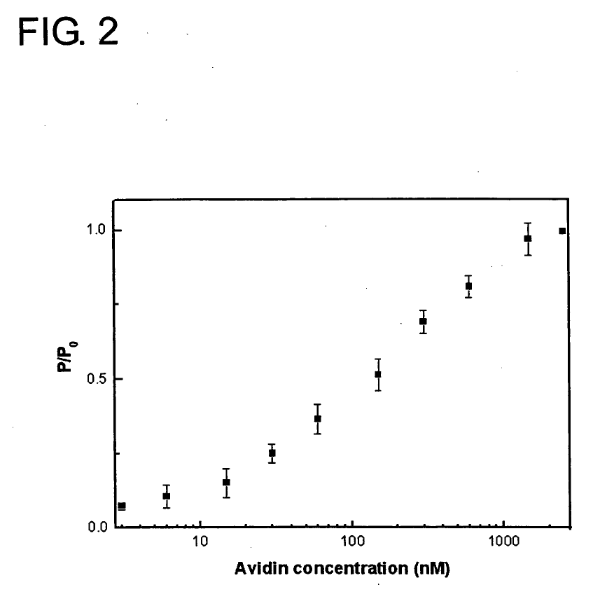

After completing the reaction, the emissions according to the concentration of avidin were measured in the experimental groups. FIG. 2 illustrates the ratio of emissions at 620 nm (the maximal emission of quantum dot). This ratio was calculated with P/Pₒ, wherein P is a value of emission in each experimental group and Pₒ, a maximal value of emission when saturated with avidins (in this case, 2.5 μM of avidin) As depicted in FIG. 2, the emission of the experimental group increased proportionally according to the concentration of avidin. Therefore, it is confirmed that the avidin included in a specimen can be detected quantitatively by estimating the ratio of emissions.

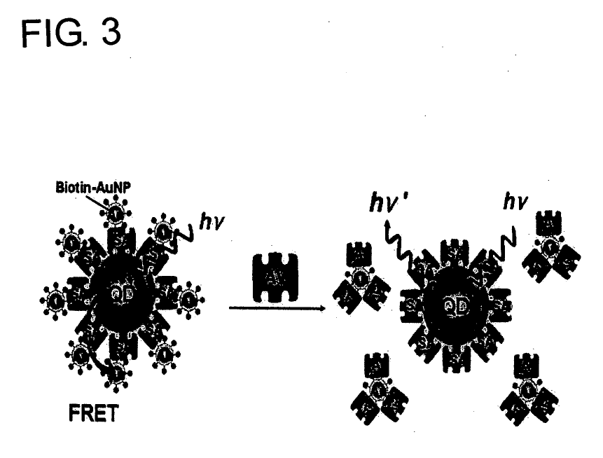

FIG. 3 depicts the conceptual diagram of mechanism that the emission of quantum dot does not decrease as the specific binding between BG nano-particle and SQ dot is inhibited by avidins. That is to say, FIG. 3 illustrates the disappearance of FRET phenomenon. If biotin and streptavidin binds specifically, the resulting emission decreased due to the FRET phenomenon between gold nano-particle and quantum dot, compared to the intrinsic emission of quantum dot. In contrast, if avidin, a target molecule also exists, the specific binding between biotin and streptavidin is inhibited and the FRET disappears. As a consequence, the decrease of emission reduces as the concentration of avidin increases.

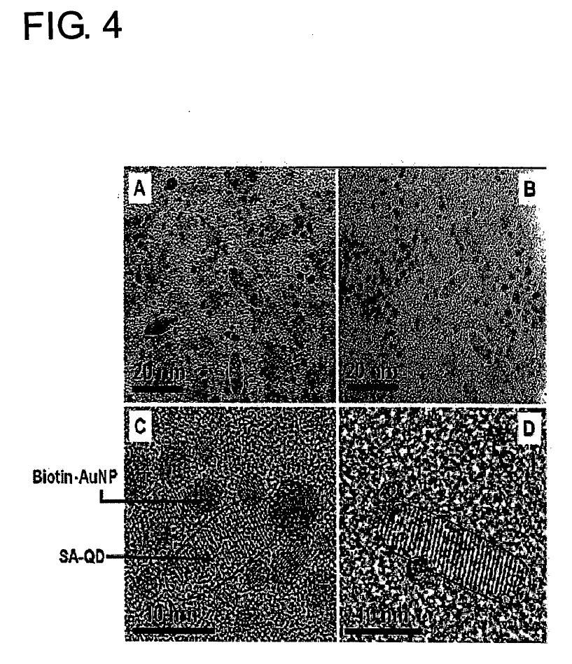

In order to elucidate this mechanism, a solution containing BG nano-particle and SQ dot was observed before and after adding avidins by performing an electron microscopy. (See FIG. 4) FIG. 4A illustrates the specific binding of BG nano-particle and SQ dot. FIG. 4B illustrates the inhibition of the specific binding when reacting BG nano-particle with avidin (3 μM) before adding SQ dot. FIGS. 4C and D illustrates a lattice structure of each metal nano-particle and a binding between metal nano-particles by magnifying FIG. 4A and B respectively.

### Example 2

### Analysis of Glycoprotein by Using LG Nano-Particle and DQ Dot

(1) Preparation of LG Nano-Particle

In order to produce LG nano-particle as a target molecule, 0.01 g of HAuCl₄.3H₂O (purchased from Aldrich Corporation) was dissolved in 10 ml of DDW to prepare a gold chloride solution. Then, 0.02 g of sodium citrate dehydrate (2-hydroxy-1,2,3-propanetricarboxylic acid was added to the gold chloride solution and stirred vigorously for a minute. After that, 1 mg of NaBH₄ was added to the resulting solution and stirred strongly for 5 minutes to prepare a red wine-colored gold nano-particle. When performing an UV/Vis spectroscopy, the gold nano-particles showed a slightly higher absorbance than adjacent bands at approximately 500˜540 nm of wavelength by surface plasmon resonance. As a result, it is confirmed that the gold nano-particle is prepared successfully.

After that, 1 ml of the gold nano-particle solution that is modified with sodium citrate on the surface as prepared by the above-mentioned procedure was mixed with 5.1 mg of concanavalin A (purchased from Sigma), a sort of lectin and stirred for 8 hours. After reacting, the solution was centrifuged or filtered to discard non-reactive proteins to prepare convanavain A conjugated gold nano-particle (hereinafter, referred to as “CG nano-particle”).

(2) Preparation of DQ Dot

Dextran (10,000 kDa) is an elongated glucose, a sort of carbohydrate binding specifically with concanavalin A.

In order to prepare DQ dot, 50 mg of dextran (Sigma) and 10 mg of 1,1′-carbonyldiimidazole(CDI)(purchased from Sigma) were dissolved in 1 ml of 50% dimethylsulfoxide(DMSO) and reacted at room temperature for 30 minutes to prepare CDI conjugated dextran. 10 μl of the CDI conjugated dextran was added to 1 ml of 80 nM solution of amine modified quantum dot (purchased from Quantum Dot Corporation) and stirred at room temperature for 24 hours. Then, the resulting solution was filtered to discard non-reactive dextran and again dissolved in DDW to prepare DQ dot solution.

(3) Analysis of Glycoprotein According to Concentration by Using FRET between LG Nano-Particle and DQ Dot

In order to examine a glycoprotein, neoglycosylated BSA having 22 mannoses, a sort of carbohydrate specifically binding with concanavalin A was purchased from Sigma. Then, an analysis of target molecules binding between CG nano-particle and DQ dot was conducted.

When performing the same procedure described in Example 1(2), it is observed that the FRET phenomenon between quantum dot and gold nano-particle occurred by a specific binding between CG nano-particle and DQ nano-particle (data not shown).

In a solution of 10 nM CG nano-particle and 1 nM DQ dot, neoglycosylated BSA containing 22 mannose residues was added and adjusted to its final concentrations in 1 nM˜10 μM. Then, 12 specimens respectively containing an inhibitor with a different concentration were prepared and stirred weakly at room temperature for an hour. In each specimen, the resulting fluorescence was measured at 605 nm.

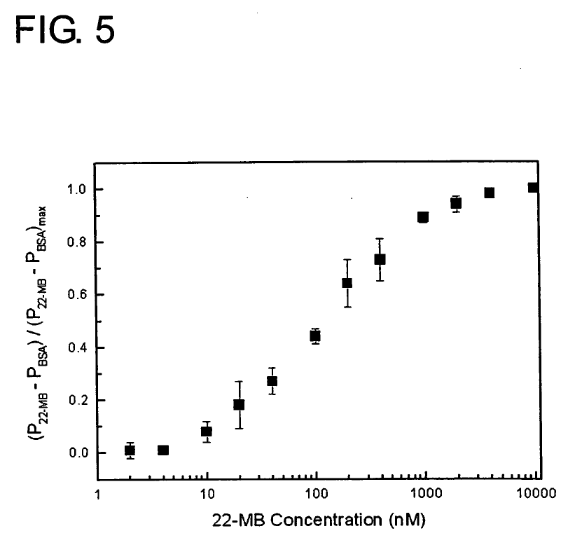

This experiment was conducted in 200 μl volume of tube and the fluorescence spectrum was obtained with a fluorometer. For a control group, a specimen adding general BSA without mannose in the same concentration was prepared. The maximal value of quantum dot emission was observed at near 605 nm. The fluorescences of the experimental group and the control group were measured at 605 nm, as illustrated in FIG. 5. For convenience, the discrepancy of emissions between the experimental group and the control group (P_{22-MB}-P_{BSA}) was calculated at each protein concentration to obtain a ratio against the maximal value (P_{22-MB}-P_{BSA})ₘₐₓ. FIG. 5 demonstrates the ratio of emissions according to the protein concentration (X-axis) ((P_{22-MB}-P_{BSA})/P_{22-MB}-P_{BSA})ₘₐₓ; Y-axis).

As a result, it is noted that when the concentration of the neoglycosylated BSA having 22 mannoses increases, the fluorescence of quantum dot is maintained highly. Concanavalin A onto the CG nano-particle is increasingly blocked to interfere a specific binding between gold nano-particle and quantum dot and the FRET phenomenon does not occur.

In FIG. 5, the emission of quantum dot increased proportionally according to the concentration of mannose on the neoglycosylated BSA. Therefore, it is also confirmed that glycoproteins can be detected quantitatively by estimating the emission ratio of quantum dots.

(4) Analysis of Glycoprotein According to the Amount of Carbohydrates by Using FRET between LG Nano-Particle and DQ Dot

In addition to the concentration of glycoprotein, the amount of carbohydrates per glycoprotein, if being different, can be analyzed at the same concentration of glycoprotein by using this system.

Above all, in order to prepare a neoglycoprotein using general BSA, the BSA without a carbohydrate was conjugated at lysine residues with commercially available α-D-mannopyranosyl-phenyl-isothiocyanate (MPI; purchased from Sigma) at NCS residues through a covalent bond. At this moment, the ratio of MPI and BSA is adjusted in 1.5:1 to 150:1 to prepare BSA conjugating mannose in a different amount.

Then, MPI was dissolved in dimethylsulfoxide (DMSO) and reacted with a solution of BSA and 20% DMSO in various ratios at 4° C. for 24 hours. After completing the reaction, the resulting solution was filtered to separate MPI conjugated neoglycoproteins. By performing a carbohydrate analysis using Bio-LC, it is identified that the resulting glycoproteins are conjugated with 0, 1.5, 2.8, 5.8, 10, 12.6, 15, 21.4, or 22 mannoses per BSA.

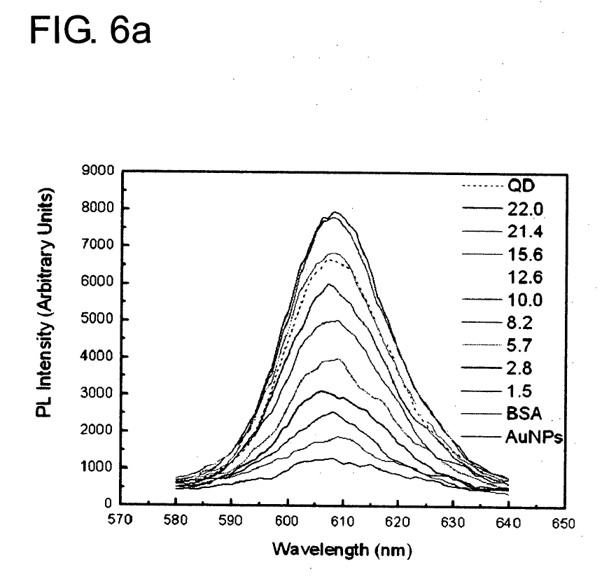

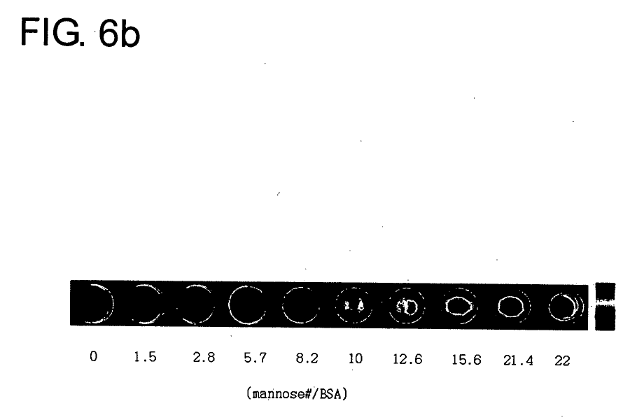

This specimen was added to the solution of CG nano-particle and DQ dot in the same amount and reacted to measure the fluorescent emission of the reactant. It is noted that when increasing the number of mannose, photoluminescence (PL: fluorescence or luminescence) is maintained highly. Concanavalin A onto the CG nano-particle is increasingly blocked to interfere a binding between CG nano-particle and DQ dot. The result is illustrated in FIG. 6a. As a consequence, it is clear that the glycoprotein can be analyzed according to the amount of carbohydrates of glycoprotein by using this quantitative system.

Furthermore, in order to establish a system, a mini-well plate having wells in 10 μl of volume was introduced to measure a number of specimens simultaneously even in a low volume. Then, the specimen was reacted coincidently and read with an image analyzer. The result is illustrated in FIG. 6b. When moving from blue color to red color, the fluorescence signal of the image result obtained by charge coupled device (CCD) using 605 nm of filter became higher.

As described in the test tube, it is noted that the strength of emission increases by the inhibition when the amount of carbohydrates in neoglycoprotein increases.

As illustrated and confirmed above, the process according to the present invention is rapid and easy without labeling a specimen using the FRET between metal nano-particle and quantum dot. This process can be applied to screen a biochemical inhibitor interfering a specific binding between a pair of bio-molecules in an ultra-high speed and measure a quantity of the substance and develop novel drugs.

In addition, this method can be used to analyze characteristics such as change of the amount of carbohydrates in a glycoprotein derived from various cells as a drug candidate and thereby, is applicable for a quality control of proteins etc.

Besides, according to the method for detecting interactive bio-molecules in the present invention, inhibitors interfering a binding between particular substances as well as target molecules may be screened in a high speed.

Furthermore, this method is possible to replace conventional hybridizations for sensing a complementary sequence of nucleic acids and reactions for detecting a target molecule by using antigen-antibody and protein-ligand etc. Therefore, this process can be applied to find out biochemical substances or bio-molecules and especially, to conduct drug-screenings efficiently.

Those skilled in the art will appreciate that the conceptions and specific embodiments disclosed in the foregoing description may be readily utilized as a basis for modifying or designing other embodiments for carrying out the same purposes of the present invention.

Those skilled in the art will also appreciate that such equivalent embodiments do not depart from the spirit and scope of the invention as set forth in the appended claims.

What is claimed is:

1. A method for detecting a specific binding for a pair of bi-molecules, which comprises steps: (1) selecting a pair of energy donor and energy acceptor that displays a FRET phenomenon when approaching in a predetermined distance, as a metal nano-particle and a quantum dot respectively; and then, conjugating a pair of bio-molecules independently at the metal nano-particle and the quantum dot; and (2) blending the resulting bio-molecule conjugated metal nano-particle (BM particle) and the bio-molecule conjugated quantum dot (BQ dot) in a liquid state; and then, identifying an occurrence of the FRET phenomenon by using a fluorescence assay.

2. The method for detecting a specific binding for a pair of bi-molecules according to claim 1, in which the metal nano-particle is selected from a group comprising gold nano-particle, silver nano-particle and platinum nano-particle.

3. The method for detecting a specific binding for a pair of bi-molecules according to claim 1, in which the pair of bio-molecules is selected from a group comprising DNA, RNA, PNA, protein, glycoprotein and carbohydrate.

4. A method for detecting a target molecule in a specimen, which comprises steps: (1) selecting a pair of energy donor and energy acceptor that displays a FRET phenomenon when approaching in a predetermined distance metal, as a metal nano-particle and a quantum dot respectively; then, conjugating a pair of bio-molecules (a bio-molecule 1 and a bio-molecule 2) at the metal nano-particle and the quantum dot respectively; and preparing a bio-molecule 1 conjugated metal nano-particle (B1M particle) and a bio-molecule 2 conjugated quantum dot (B2Q dot); (2) blending the B1M particle and the B2Q dot in a liquid state; and then, identifying a specific binding for the B1M particle and the B2Q dot; and (3) blending the B1M particle and the B2Q dot with a specimen; and then, analyzing a change of the FRET occurrence by using a fluorescence assay.

5. The method for detecting a target molecule in a specimen according to claim 4, in which the metal nano-particle is selected from a group comprising gold nano-particle, silver nano-particle and platinum nano-particle.

6. The method for detecting a target molecule in a specimen according to claim 4, in which the pair of bio-molecules is selected from a group comprising DNA, RNA, PNA, protein, glycoprotein and carbohydrate.

7. A method for measuring a quantity of target molecules in a specimen, which comprises steps: (1) selecting a pair of energy donor and energy acceptor that displays a FRET phenomenon when approaching in a predetermined distance metal, as a metal nano-particle and a quantum dot respectively; then, conjugating a pair of bio-molecules (a bio-molecule 1 and a bio-molecule 2) at the metal nano-particle and the quantum dot respectively; and preparing a bio-molecule 1 conjugated metal nano-particle (B1M particle) and a bio-molecule 2 conjugated quantum dot (B2Q dot); (2) blending the B1M particle and the B2Q dot in a liquid state; and then, identifying a specific binding for the B1M particle and the B2Q dot to maintain the specific binding; and (3) blending the B1M particle and the B2Q dot with a specimen containing a target molecule inhibiting a specific binding between the bio-molecule 1 and the bio-molecule 2; and then, analyzing a degree of the change in the FRET occurrence by using a fluorescence assay.

8. The method for measuring a quantity of target molecules in a specimen according to claim 7, in which the metal nano-particle is selected from a group comprising gold nano-particle, silver nano-particle and platinum nano-particle.

9. The method for measuring a quantity of target molecules in a specimen according to claim 7, in which the pair of bio-molecules is selected from a group comprising DNA, RNA, PNA, protein, glycoprotein and carbohydrate.
